(19) United States
(12) Patent Application Publication
(10) Pub. No.: US 2024/0418682 A1
(43) Pub. Date: Dec. 19, 2024

(54) AUTO TRAJECTORY CORRECTION FOR NON-DESTRUCTIVE TEST

(71) Applicant: Evident Canada, Inc., Québec (CA)

(72) Inventors: Dominique Nogues, Québec (CA); Etienne Grondin, Québec (CA); Yixin Liu, Québec (CA)

(21) Appl. No.: 18/703,052

(22) PCT Filed: Oct. 21, 2022
(86) PCT No.: PCT/CA2022/051559

§ 371 Date: Apr. 19, 2024

Related U.S. Application Data
(60) Provisional application No. 63/262,847, filed on Oct. 21, 2021.

(51) Int. Cl. G01N 29/265; G01N 29/07; G01N 29/22

(52) U.S. Cl. CPC G01N 29/265; G01N 29/07; G01N 29/221; G01N 29/225; G01N 2291/011

(57) ABSTRACT

Apparatus and techniques described herein can be used to compensate for variation in flatness or other surface features of a planar or nearly-planar test specimen, such as facilitating acoustic inspection. According to examples herein, compensation can be performed such as to maintain a parallel orientation of a non-destructive test probe relative to a surface of a test specimen or to maintain a specified distance between the test probe and the surface, or both. Such an approach can include use of multiple probe elements such as to contemporaneously acquire data indicative of a surface profile of the test specimen (such as using a time-of-flight determination), and to perform an inspection acquisition. In this manner, a probe trajectory can be adjusted (e.g., updated) during an acquisition to enhance inspection productivity versus other approaches.

### CLAIM OF PRIORITY

This patent application claims the benefit of priority of Dominique Nogues et al., U.S. Provisional Patent Application Ser. No. 63/262,847, titled “AUTO TRAJECTORY CORRECTION FOR ACCURATE INSPECTION,” filed on Oct. 21, 2021 (Attorney Docket No. 6409.217PRV), which is hereby incorporated by reference herein in its entirety.

### FIELD OF THE DISCLOSURE

This document pertains generally, but not by way of limitation, to apparatus and techniques for non-destructive inspection such as facilitating acoustic inspection, and more particularly, to apparatus and techniques for determining a profile of a surface of an object under inspection and adapting a probe orientation to follow such a surface.

### BACKGROUND

Non-destructive testing (NDT) can refer to use of one or more different techniques to inspect regions on or within an object, such as to ascertain whether flaws or defects exist, or to otherwise characterize the object being inspected. Examples of non-destructive test approaches can include use of an eddy-current testing approach where electromagnetic energy is applied to the object and resulting induced currents on or within the object are detected, with the values of a detected current (or a related impedance) providing an indication of the structure of the object under test, such as to indicate a presence of a crack, void, porosity, or other inhomogeneity.

Another approach for NDT can include use of an acoustic inspection technique, such as where one or more electroacoustic transducers are used to insonify a region on or within the object under test, and acoustic energy that is scattered or reflected can be detected and processed. Such scattered or reflected energy can be referred to as an acoustic echo signal. Generally, such an acoustic inspection scheme involves use of acoustic frequencies in an ultrasonic range of frequencies, such as including pulses having energy in a specified range that can include value from, for example, a few hundred kilohertz, to tens of megahertz, as an illustrative example.

### SUMMARY OF THE DISCLOSURE

Non-destructive test (NDT) can include apparatus and techniques for inspecting various objects, such as using optical, acoustic, or electromagnetic techniques, or combinations thereof. For example, a test specimen such as a steel plate, aluminum structure, or composite structure can be inspected using an acoustic inspection technique. Generally, even if multiple inspection probes are used, a single probe is too small to achieve coverage of an entirety of such a test specimen. Accordingly, in order to achieve specified test coverage, a scanning approach is used where a test probe or a test specimen are moved relative to each other in a manner to provide such scanning.

The present inventors have, among other things, developed apparatus and techniques that can be used to compensate for variation in flatness or other surface features of a planar or nearly-planar test specimen, such as facilitating acoustic inspection. According to examples herein, compensation can be performed such as to maintain a parallel orientation of a non-destructive test probe relative to a surface of a test specimen or to maintain a specified distance between the test probe and the surface, or both. Such an approach can include use of multiple probe elements such as to contemporaneously acquire data indicative of a surface profile of the test specimen (such as using a time-of-flight determination), and to perform an inspection acquisition. In this manner, a probe trajectory can be adjusted (e.g., updated) during an acquisition to enhance inspection productivity versus other approaches.

In an example, a machine-implemented method for performing non-destructive test (NDT) including compensating for surface variation of a test specimen can include positioning a probe assembly relative to a surface of the test specimen to perform scanning. At a first scan location, the method can include acquiring data indicative of a distance between the probe assembly and the surface of the test specimen using a first transducer arrangement of the probe assembly, determining a corrected probe assembly orientation using the data indicative of the distance, and at the first scan location, performing an inspection acquisition using a second transducer arrangement and using the corrected probe assembly orientation, the inspection acquisition separate from an acquisition used for a determination of the distance using the first transducer arrangement.

In an example, a system for performing non-destructive test (NDT) including compensating for surface variation of a test specimen can include a probe assembly, a manipulator mechanically coupled with the probe assembly and configured to position and orient the probe assembly, a processor circuit, and a memory circuit comprising instructions that, when executed by the processor circuit, cause the system to position the probe assembly relative to a surface of the test specimen, using the manipulator, to perform scanning. At a first scan location, data indicative of a distance between the probe assembly and the surface of the test specimen can be acquired. A corrected probe assembly orientation can be determined using the data indicative of the distance, and at the first scan location, an inspection acquisition can be performed using the corrected probe assembly orientation, the inspection acquisition separate from an acquisition used for a determination of the distance using the first transducer arrangement.

In an example, the probe assembly mentioned in the examples of above comprises an acoustic probe assembly, the acoustic probe assembly comprising a first region defining the first transducer arrangement, and a second region defining the second transducer arrangement. In an example, the acoustic probe assembly comprises a linear array of electroacoustic transducer elements. In an example, establishing the corrected probe assembly orientation includes tilting the probe assembly in at least one axis. In an example, acquiring data indicative of the distance comprises performing an acoustic time-of-flight (ToF) determination, such as using an acquired echo indicative of a front wall of a test specimen.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates generally an example comprising an acoustic inspection system, such as can be used to perform at least a portion one or more techniques as shown and described herein.

FIG. 2 illustrates generally an example comprising a system for non-destructive inspection, such as can be used to perform at least a portion one or more techniques as shown and described herein.

FIG. 3 illustrates generally an example comprising a probe assembly, such as can be used to perform at least a portion of one or more techniques shown and described herein.

FIG. 4 illustrates generally an example comprising a scan path and related techniques for performing non-destructive inspection, such as can be performed using the probe assembly configuration shown in FIG. 3.

FIG. 5 shows a technique, such as a machine implemented method, for performing non-destructive inspection, such as can include compensating for variations in surface planarity or flatness.

FIG. 6 shows a technique, such as a machine implemented method, for performing non-destructive inspection, such as can acquiring data indicative of a distance between a probe assembly and a surface of a test specimen, and using the acquired data to correct at least one of a probe assembly orientation or a probe assembly height.

FIG. 7 illustrates a block diagram of an example comprising a machine upon which any one or more of the techniques (e.g., methodologies) discussed herein may be performed.

### DETAILED DESCRIPTION

Non-destructive testing can be performed using various techniques such as involving acoustic, electromagnetic, or optical scanning. Generally, scanning is performed using a probe assembly that is movable relative to a test specimen (or vice versa), to achieve coverage of a portion or an entirety of the test specimen. For example, an acoustic probe assembly can be controlled using a manipulator such as a robotic manipulator or gantry. The acoustic probe assembly can be positioned at a specified nominal distance from a surface of a planar test specimen where a face or active surface of the probe is oriented parallel to a nominal surface profile of the test specimen. The manipulator can scan the acoustic probe assembly along a specified scan path according to a scan plan. The scan plan can be implemented using a variety of approaches. For example, a linear or raster scan plan can involve sweeping the acoustic probe assembly along a surface of a test specimen in a linear fashion. A line or row can scanned and then the probe assembly can be moved or offset in an orthogonal direction along the surface being scanned (e.g., “re-indexing” the probe), for performing another scan parallel to the prior line or row, until a specified area is covered. For acoustic inspection, each row in a raster scan generally includes a series of A-scan acquisitions such as for assembly of a C-scan image. The present inventors have recognized, among other things, that scanning of a test specimen with variable flatness or planarity can present challenges, even if the test specimen is nominally planar or flat.

For example, the present inventors have recognized, among other things, that a surface of the test specimen is generally not perfectly planar or flat (either due to inherent variations, defects, manufacturing process, or being intentionally contoured). Variation causing a lack of parallel orientation between the acoustic probe active surface and test specimen, or distance variation, can affect acoustic inspection results, such as invalidating an acquisition or suppressing detection of flaws. To help compensate for such variation, two scan iterations can be performed. A first scan can include scanning the acoustic probe or another sensor along the surface of the test specimen to perform a distance measurement to identify a location of a front wall of the test specimen relative to the acoustic probe, such as using a time-of-flight determination. A series of distance measurement can allow determination of a surface profile of the test specimen. Another scan can then be performed including compensating for variation from the nominal probe orientation or distance, or both, using data acquired from the first scan. Such an approach can still present various draw backs. For example, such an approach may involve two entire scan iterations performed separately and serially, such as decreasing inspection productivity or throughput.

The present inventors have recognized, among other things, that another approach can include acquiring data indicative of a probe distance from the test specimen using a portion of an acoustic probe array or transducer assembly that is located on the same assembly as a portion of the acoustic probe array that is used for inspection acquisition. For example, a first transducer arrangement of the acoustic probe assembly can be used to acquire distance data, such as using a pulse-echo acquisition or other technique for time-of-flight determination. A second transducer arrangement of the acoustic probe assembly can be used to perform inspection using a corrected probe orientation or corrected probe height (or both) based on the distance data. In this approach, the portion of the acoustic probe assembly that is used for distance determination can be used to acquire further (e.g., updated) distance data for upcoming inspection locations contemporaneously while the inspection acquisition portion of the probe assembly is used for performing inspection acquisition. For example, a first linear scan row in a scan plan can be acoustically inspected using a corrected probe orientation from a prior distance determination, while the next linear scan row is contemporaneously being measured for updating the probe orientation when the next linear scan row is inspected. In this manner, two entirely separate scan passes are not needed because a single pass can be used for performing distance determination and trajectory correction, such as in adjacent rows, as shown and described further below. As discussed below, updating of probe orientation can include rotating the probe about a first axis that is parallel to a scan direction (e.g., the “active” axis) or rotating the probe about a second axis that is orthogonal to the scan direction (e.g., the “passive” axis), or both.

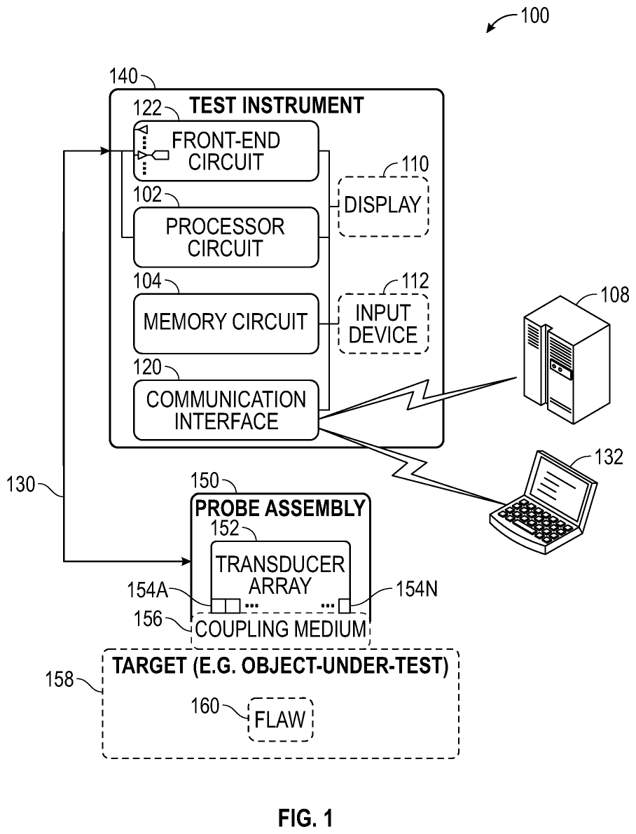

FIG. 1 illustrates generally an example comprising an acoustic inspection system 100, such as can be used to perform at least a portion one or more techniques as shown and described herein. The inspection system 100 can include a test instrument 140, such as a hand-held or portable assembly. The test instrument 140 can be electrically coupled to a probe assembly 150, such as using a multi-conductor interconnect 130. The probe assembly 150 can include one or more electroacoustic transducers, such as a transducer array 152 including respective transducers 154A through 154N. The transducers array can follow a linear or curved contour or can include an array of elements extending in two axes, such as providing a matrix of transducer elements. The elements need not be square in footprint or arranged along a straight-line axis. Element size and pitch can be varied according to the inspection application.

A modular probe assembly 150 configuration can be used, such as to allow a test instrument 140 to be used with various different probe assemblies. Generally, the transducer array 152 includes piezoelectric transducers, such as can be acoustically coupled to a target 158 (e.g., a test specimen or “object-under-test”) through a coupling medium 156. The coupling medium can include a fluid or gel or a solid membrane (e.g., an elastomer or other polymer material), or a combination of fluid, gel, or solid structures. For example, an acoustic transducer assembly can include a transducer array coupled to a wedge structure comprising a rigid thermoset polymer having known acoustic propagation characteristics (for example, Rexolite® available from C-Lec Plastics Inc.), and water can be injected between the wedge and the structure under test as a coupling medium 156 during testing, or testing can be conducted with an interface between the probe assembly 150 and the target 158 otherwise immersed in a coupling medium.

The test instrument 140 can include digital and analog circuitry, such as a front-end circuit 122 including one or more transmit signal chains, receive signal chains, or switching circuitry (e.g., transmit/receive switching circuitry). The transmit signal chain can include amplifier and filter circuitry, such as to provide transmit pulses for delivery through an interconnect 130 to a probe assembly 150 for insonification of the target 158, such as to image or otherwise detect a flaw 160 on or within the target 158 structure by receiving scattered or reflected acoustic energy elicited in response to the insonification.

While FIG. 1 shows a single probe assembly 150 and a single transducer array 152, other configurations can be used, such as multiple probe assemblies connected to a single test instrument 140, or multiple transducer arrays 152 used with a single probe assembly 150 or multiple probe assemblies for pitch/catch inspection modes. Similarly, a test protocol can be performed using coordination between multiple test instruments 140, such as in response to an overall test scheme established from a master test instrument 140 or established by another remote system such as a compute facility 108 or general-purpose computing device such as a laptop 132, tablet, smart-phone, desktop computer, or the like. The test scheme may be established according to a published standard or regulatory requirement and may be performed upon initial fabrication or on a recurring basis for ongoing surveillance, as illustrative examples.

The receive signal chain of the front-end circuit 122 can include one or more filters or amplifier circuits, along with an analog-to-digital conversion facility, such as to digitize echo signals received using the probe assembly 150. Digitization can be performed coherently, such as to provide multiple channels of digitized data aligned or referenced to each other in time or phase. The front-end circuit can be coupled to and controlled by one or more processor circuits, such as a processor circuit 102 included as a portion of the test instrument 140. The processor circuit can be coupled to a memory circuit, such as to execute instructions that cause the test instrument 140 to perform one or more of acoustic transmission, acoustic acquisition, processing, or storage of data relating to an acoustic inspection, or to otherwise perform techniques as shown and described herein. The test instrument 140 can be communicatively coupled to other portions of the system 100, such as using a wired or wireless communication interface 120.

For example, performance of one or more techniques as shown and described herein can be accomplished on-board the test instrument 140 or using other processing or storage facilities such as using a compute facility 108 or a general-purpose computing device such as a laptop 132, tablet, smart-phone, desktop computer, or the like. For example, processing tasks that would be undesirably slow if performed on-board the test instrument 140 or beyond the capabilities of the test instrument 140 can be performed remotely (e.g., on a separate system), such as in response to a request from the test instrument 140. Similarly, storage of imaging data or intermediate data such as A-scan matrices of time-series data or other representations of such data, for example, can be accomplished using remote facilities communicatively coupled to the test instrument 140. The test instrument can include a display 110, such as for presentation of configuration information or results, and an input device 112 such as including one or more of a keyboard, trackball, function keys or soft keys, mouse-interface, touch-screen, stylus, or the like, for receiving operator commands, configuration information, or responses to queries.

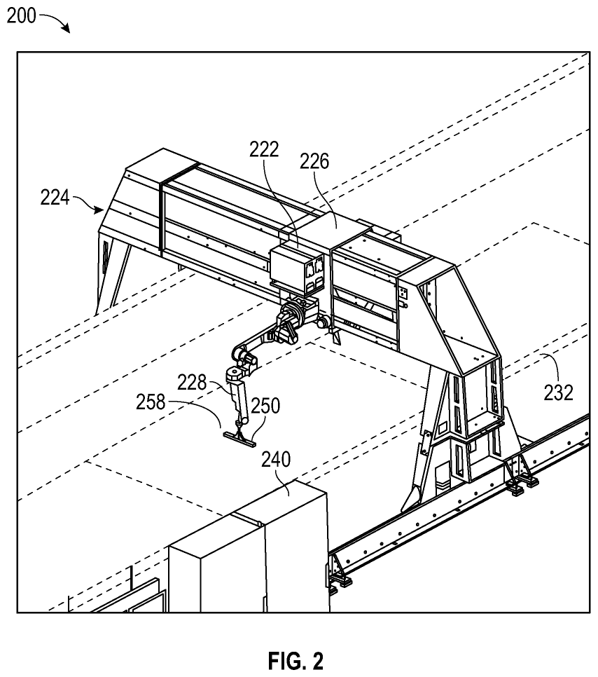

FIG. 2 illustrates generally an example comprising a system 200 for non-destructive inspection, such as can be used to perform at least a portion one or more techniques as shown and described herein. In the example of FIG. 2, a gantry 224 or other manipulator can be used to position a probe assembly 250 relative to a test specimen 258, such as a steel plate or other structure. For acoustic inspection, the steel plate can be immersed in a coupling medium such as water, in an immersion tank 232. Alternatively, or in addition, couplant can be circulated at or near the probe assembly 250 to facilitate coupling of acoustic energy to or from the test specimen 258. In the example of FIG. 2, the gantry 224 can include a carriage 226 coupled to a robotic manipulator 228, such as to support multiple degrees of freedom including rotational and translation degrees of freedom. In this manner, an orientation and position of the probe assembly 250 can be controlled. Alternatively, or in addition, the test specimen 258 can be moved relative to the probe assembly 250 such as using a conveyor or other equipment.

Acoustic inspection control circuitry 222 can be included, such as to provide an analog front end nearby the probe assembly 250 to generate signals driving electroacoustic transducers or to receive, amplify, and digitize signals received by electroacoustic transducers (or both), where the electroacoustic transducers are included as a portion of the probe assembly 250. As shown and described herein, acoustic signals can be used for a distance determination to provide a corrected probe assembly 250 orientation or corrected probe assembly 250 height (or both) relative to the test specimen 258. For example, a pulse-echo or other time-of-flight measurement can be performed using the probe assembly 250. Data acquired using the probe assembly 250 can be archived, processed, or presented such as using test instrumentation 240 located at or nearby the system 200, or located elsewhere. The system 200 can be included as an element in a production line where the test specimen 258 is fabricated or is to be used in downstream operations. The present inventors have recognized that for acoustic inspection of a small defects in a flat plate such as the test specimen 258 shown in FIG. 2, such inspection is potentially a slow process that can include use of multiple passes while the test specimen 258 is immersed in the immersion tank 232. If the test specimen 258 is not perfectly flat or planar, manual realignment of the probe assembly 250 may be performed by an operator, to achieved desired coverage of an entirety of the test specimen 258, such as involving multiple scan passes. The present inventors have recognized, among other things, that a distance determination can be performed to provide a corrected probe assembly 250 orientation or a corrected probe assembly 250 height, or both, such as in an automated or semi-automated manner, and using as few as a single scan pass.

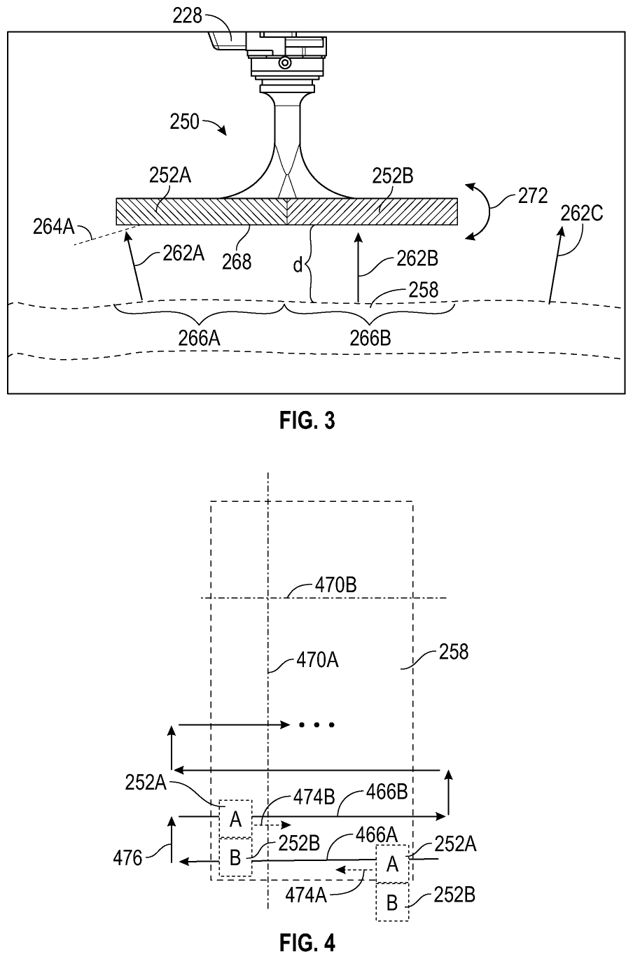

As an illustration, FIG. 3 illustrates generally an example comprising a detailed view of a probe assembly 250 such as located at or otherwise serving as an end effector of a robotic manipulator 228. The configuration shown in FIG. 3 can be used to perform at least a portion of one or more techniques shown and described herein. In the example of FIG. 3, the probe assembly 250 can include two portions, such as a first transducer arrangement 252A and a second transducer arrangement 252B. For example, the first transducer arrangement 252A can include an array of electroacoustic transducer elements, such as a linear array, and the second transducer arrangement 252B can include another array of electroacoustic transducer elements (or the arrangements 252A and 252B can be sub-arrays of a single array). If a surface of the test specimen 258 is not perfectly flat, such as curved (exaggerated) as shown in FIG. 3, then inspection results can be affected (e.g., defects or flaws may be missed) if an active surface 268 of the probe assembly 250 is not oriented parallel to the surface of the test specimen 258. As an illustration, a first surface normal vector 262A can be defined corresponding to a region 266A, a second surface normal vector 262B can be defined corresponding to a region 266B, elsewhere another surface normal vector 262C can be defined, and so on. A mapping of surface flatness can be used to establish a corrected probe assembly 250 orientation or height, such as using acoustic transducers to perform distance determination or using another technique (e.g., an optical sensor located on or otherwise coupled to the probe assembly 250).

As similarly shown and described below in FIG. 4., in FIG. 3 the first transducer arrangement 252A can be used to acquire data indicative of a distance, “d” of the probe assembly 250 from the test specimen 258, such as during a first pass of a linear scan row in a scan plan, and a probe orientation can be corrected. During a second pass of the linear scan row; the second transducer arrangement 252B is aligned (such as tilted as shown by arrows 272) to compensate for variation in surface flatness for an acoustic inspection and passes over the region 266B that was previously gauged for probe distance. In this manner, a surface profile can be established and used to maintain parallelism between the active surface 268 of the probe assembly 250 and the test specimen 258 to within a specified angular range, such as within a range of plus or minus 1 degree, or plus or minus 0.5 degrees.

In an example, when the second transducer arrangement 252B is performing an acoustic inspection acquisition, the first transducer arrangement 252A can be contemporaneously acquiring data indicative of a distance between the probe assembly 250 and the test specimen 258 to update the probe assembly 250 orientation or height when the second transducer arrangement 252B is re-indexed to perform an acoustic inspection acquisition in the region 266A. For example, the probe assembly 250 can be tilted counter-clockwise to orient the probe assembly 250 active surface 268 in a manner parallel to the test specimen 258 as shown by the line 264A and orthogonal to a determined surface normal vector 262A. The probe assembly 250 can also be controlled using acquired distance data to provide a nominal distance, “d,” for acoustic inspection acquisition, by raising or lowering the probe assembly 250 in addition to tilting the probe assembly, such as to provide a consistent couplant column (e.g., water column) thickness between the active surface 268 and the test specimen 258. In addition to the tilting as shown by arrows 272 (or instead), tilting can be performed in other axes, such as orthogonally to the plane of the drawing of FIG. 3. In this manner, control of probe orientation via tilting need not be restricted to rotation about a single axis and can include rotation of the probe about a first axis that is parallel to a scan direction (e.g., the “active” axis) or rotating the probe about a second axis that is orthogonal to the scan direction (e.g., the “passive” axis), or both, as illustrative examples.

As mentioned above, various approaches can be used to implement a scan plan or scan path to achieve desired coverage of the specimen 258. As an illustration, FIG. 4 illustrates generally an example comprising a scan path and related techniques for performing non-destructive inspection, such as can be performed using the probe assembly configuration shown in FIG. 3. In the example of FIG. 4, coverage of an entirety of a surface of the test specimen 258, or at least extending to one or more edges of the test specimen 258, can be achieved by extending a portion of the test probe assembly outside or otherwise beyond a footprint of the test specimen 258.

For example, during a first pass in direction 474A, a first transducer arrangement 252A, “A,” can be scanned along a segment 466A of a scan path corresponding to a row in a raster scan, such as to acquire distance data indicative of a separation between the probe assembly and the test specimen 258. Such a scan along segment 466A can provide data indicative of a surface profile in an axis parallel to the line 470B, though the test specimen 258 may also vary along an orthogonal axis defined by line 470A. The probe assembly can then be re-indexed along line 476 to be set up for acquisition of distance data along segment 466B by the first transducer arrangement 252A. After such re-indexing, a second transducer arrangement 252B, “B,” can be scanned along the prior segment 466A in direction 474B with an updated probe orientation, or probe height, or both, such as to perform an acoustic inspection acquisition separate from distance gauging. Because the first and second transducer arrangements 252A and 252B can be part of the same probe assembly, the inspection acquisition along the segment 466A by the second transducer arrangement 252B can be performed contemporaneously with acquisition of distance data by the first transducer arrangement 252A as the probe performs the next pass in the direction 474B. In this manner, distance gauging and inspection acquisition can be performed in a single overall scan plan. Alternatively, a portion or an entirety of the test specimen 258 could be scanned for purposes of acquiring distance data and establishing a corresponding surface profile, and a separate scan iteration can be performed using an updated probe orientation or height (or both) to provide a compensated probe trajectory. Such an approach can be more time consuming, because the probe assembly may be stepped through each segment 466A, 466B, and so on, for distance gauging, and then stepped through the same series of segments 466A, 466B, and so on, for acoustic inspection acquisition using a compensated probe trajectory.

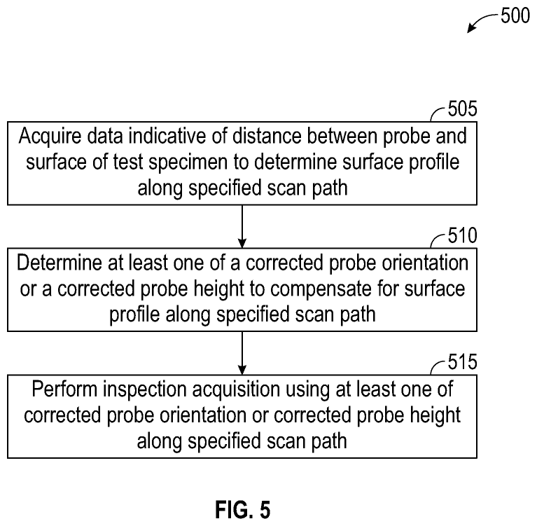

FIG. 5 shows a technique, such as a machine implemented method 500, for performing non-destructive inspection, such as can include compensating for variations in surface planarity or flatness, such as using a portion or an entirety of the apparatus and techniques discussed above in relation to FIG. 1, FIG. 2, FIG. 3, or FIG. 4, or combinations thereof. In the example of FIG. 5, the method 500 can include at 505 acquiring data indicative of a distance between a probe assembly, such as an acoustic inspection probe assembly, and a surface of a test specimen. In this manner, a surface profile can be determined along a scan path, or along at least a portion of a scan path such as along a segment defining a row corresponding to a linear acquisition of one or more A-scan representations. At 510, at least one of a corrected probe assembly orientation or a corrected probe assembly height can be determined, such as to compensate for variation a surface profile along the scan path. Such variation can include flatness or thickness variation, for example. At 515, an inspection acquisition can be performed using the at least one of the corrected probe assembly orientation or the corrected probe height (or both), along the specified scan path.

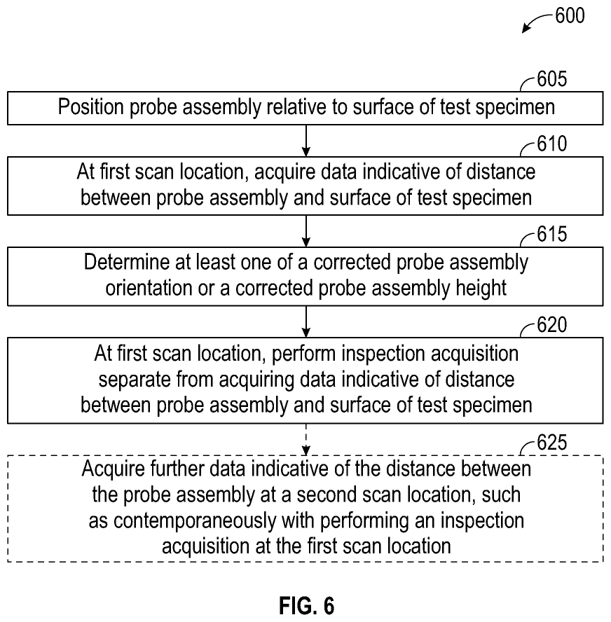

FIG. 6 shows a technique, such as a machine implemented method 600, for performing non-destructive inspection, such as can acquiring data indicative of a distance between a probe assembly and a surface of a test specimen, and using the acquired data to correct at least one of a probe assembly orientation or a probe assembly height, such as using a portion or an entirety of the apparatus and techniques discussed above in relation to FIG. 1, FIG. 2, FIG. 3, or FIG. 4, or combinations thereof. At 605, a probe assembly can be positioned relative to a surface of a test specimen, such as where the test specimen is moved via conveyor to a region where inspection can be performed, or using a gantry or other manipulator, or a combination of techniques. At 610, such as at a first scan location (e.g., along a first row in a linear or raster scan plan), data can be acquired indicative of a distance between the probe assembly and a surface of the test specimen, such as for use in determining a surface profile of the test specimen. At 615, at least one of a corrected probe assembly orientation or a corrected probe height can be determined, using the data acquired at 610. At 620, an inspection acquisition can be performed, using the corrected probe assembly orientation or the corrected probe height, or both. As shown and described above, the acquisition of data indicative of distance at 610 can be performed using a first transducer arrangement, and the inspection acquisition (e.g., for defect identification) can be performed using a second transducer arrangement. Generally, the inspection acquisition operation at 620 is separate from the acquisition of data indicative of the distance at 610. Optionally, at 625, further data can be acquired indicative of the distance between the probe assembly and the test specimen at a second scan location. For example, such acquisition at 625 can be performed along a row adjacent to a row where the first scan location is established. As shown and described in other examples, such acquisition at 625 at the second scan location can be performed contemporaneously with the inspection acquisition at the first scan location, such as by using a probe assembly having two array portions defining the first and second transducer arrangements, respectively.

Generally, in the examples described in this document, a probe assembly trajectory can be corrected to compensate for variations in flatness or planarity, such as by determining a corrected probe assembly trajectory in advance of an inspection acquisition, or “on-the-fly” such as on a row-by-row basis. Use of a corrected probe assembly trajectory can enhance defect coverage or otherwise enhance inspection productivity. If a distance measurement is being performed with a probe assembly in an orientation that has already been corrected for a prior row or scan path, the distance data acquisition can be considered an “update” such as providing further indicia of the surface profile that can be used to further update the probe assembly orientation. In this manner, it is not required to orient the probe assembly back to a nominal orientation in order to perform further distance data acquisitions.

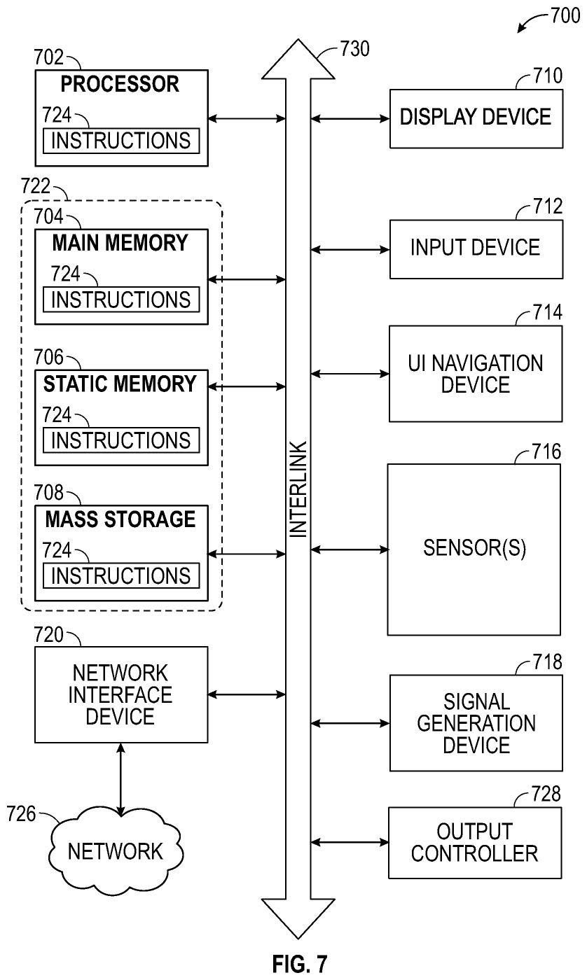

FIG. 7 illustrates a block diagram of an example comprising a machine 700 upon which any one or more of the techniques (e.g., methodologies) discussed herein may be performed. Machine 700 (e.g., computer system) may include a hardware processor 702 (e.g., a central processing unit (CPU), a graphics processing unit (GPU), a hardware processor core, or any combination thereof), a main memory 704 and a static memory 706, connected via an interlink 730 (e.g., link or bus), as some or all of these components may constitute hardware for systems or related implementations discussed above.

Specific examples of main memory 704 include Random Access Memory (RAM), and semiconductor memory devices, which may include storage locations in semiconductors such as registers. Specific examples of static memory 706 include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; RAM; or optical media such as CD-ROM and DVD-ROM disks.

The machine 700 may further include a display device 710, an input device 712 (e.g., a keyboard), and a user interface (UI) navigation device 714 (e.g., a mouse). In an example, the display device 710, input device 712, and UI navigation device 714 may be a touch-screen display. The machine 700 may include a mass storage device 708 (e.g., drive unit), a signal generation device 718 (e.g., a speaker), a network interface device 720, and one or more sensors 716, such as a global positioning system (GPS) sensor, compass, accelerometer, or some other sensor. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The mass storage device 708 may comprise a machine-readable medium 722 on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the mass storage device 708 comprises a machine readable medium.

Specific examples of machine-readable media include, one or more of non-volatile memory, such as semiconductor memory devices (e.g., EPROM or EEPROM) and flash memory devices; magnetic disks, such as internal hard disks and removable disks; magneto-optical disks; RAM; or optical media such as CD-ROM and DVD-ROM disks. While the machine-readable medium is illustrated as a single medium, the term “machine readable medium” may include a single medium or multiple media (e.g., a centralized or distributed database, or associated caches and servers) configured to store the one or more instructions 724.

An apparatus of the machine 700 includes one or more of a hardware processor 702 (e.g., a central processing unit (CPU), a graphics processing unit (GPU), a hardware processor core, or any combination thereof), a main memory 704 and a static memory 706, sensors 716, network interface device 720, antennas, a display device 710, an input device 712, a UI navigation device 714, a mass storage device 708, instructions 724, a signal generation device 718, or an output controller 728. The apparatus may be configured to perform one or more of the methods or operations disclosed herein.

The term “machine readable medium” includes, for example, any medium that is capable of storing, encoding, or carrying instructions for execution by the machine 700 and that cause the machine 700 to perform any one or more of the techniques of the present disclosure or causes another apparatus or system to perform any one or more of the techniques, or that is capable of storing, encoding or carrying data structures used by or associated with such instructions. Non-limiting machine-readable medium examples include solid-state memories, optical media, or magnetic media. Specific examples of machine-readable media include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); or optical media such as CD-ROM and DVD-ROM disks. In some examples, machine readable media includes non-transitory machine-readable media. In some examples, machine readable media includes machine readable media that is not a transitory propagating signal.

The instructions 724 may be transmitted or received, for example, over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®), IEEE 802.15.4 family of standards, a Long Term Evolution (LTE) 4G or 5G family of standards, a Universal Mobile Telecommunications System (UMTS) family of standards, peer-to-peer (P2P) networks, satellite communication networks, among others.

In an example, the network interface device 720 includes one or more physical jacks (e.g., Ethernet, coaxial, or other interconnection) or one or more antennas to access the communications network 726. In an example, the network interface device 720 includes one or more antennas to wirelessly communicate using at least one of single-input multiple-output (SIMO), multiple-input multiple-output (MIMO), or multiple-input single-output (MISO) techniques. In some examples, the network interface device 720 wirelessly communicates using Multiple User MIMO techniques. The term “transmission medium” shall be taken to include any intangible medium that is capable of storing, encoding or carrying instructions for execution by the machine 700, and includes digital or analog communications signals or other intangible medium to facilitate communication of such software.

### Various Notes

Each of the non-limiting aspects above can stand on its own or can be combined in various permutations or combinations with one or more of the other aspects or other subject matter described in this document.

The above detailed description includes references to the accompanying drawings, which form a part of the detailed description. The drawings show, by way of illustration, specific embodiments in which the invention can be practiced. These embodiments are also referred to generally as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc., are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Such instructions can be read and executed by one or more processors to enable performance of operations comprising a method, for example. The instructions are in any suitable form, such as but not limited to source code, compiled code, interpreted code, executable code, static code, dynamic code, and the like. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A machine-implemented method for performing non-destructive test (NDT) including compensating for surface variation of a test specimen, the method comprising:
positioning a probe assembly relative to a surface of the test specimen to perform scanning;
at a first scan location, acquiring data indicative of a distance between the probe assembly and the surface of the test specimen using a first transducer arrangement of the probe assembly;
determining a corrected probe assembly orientation using the data indicative of the distance; and
at the first scan location, performing an inspection acquisition using a second transducer arrangement and using the corrected probe assembly orientation, the inspection acquisition separate from an acquisition used for a determination of the distance using the first transducer arrangement.

2. The machine-implemented method of claim 1, wherein the probe assembly comprises an acoustic probe assembly, the acoustic probe assembly comprising a first region defining the first transducer arrangement, and a second region defining the second transducer arrangement.

3. The machine-implemented method of claim 2, wherein the acoustic probe assembly comprises a linear array of electroacoustic transducer elements.

4. The machine-implemented method of claim 1, comprising acquiring further data indicative of the distance between the probe assembly and the surface using the first transducer arrangement at a second scan location, when the second transducer arrangement is positioned to perform the inspection acquisition at the first scan location.

5. The machine-implemented method of claim 4, wherein acquiring the further data indicative of the distance is performed contemporaneously with performing the inspection acquisition at the first scan location.

6. The machine-implemented method of claim 4, comprising repositioning the probe assembly to provide a further corrected probe assembly orientation using the further data indicative of the distance; and
performing an inspection acquisition using the second transducer arrangement at the second scan location, using the further corrected probe assembly orientation.

7. The machine-implemented method of claim 1, wherein the data indicative of the distance comprises data indicative of a surface profile of the test specimen.

8. The machine-implemented method of claim 7, wherein the surface profile is determined using a series of distance determinations.

9. The machine-implemented method of claim 7, wherein the data indicative of the distance comprises a determination of a vector that is orthogonal to the surface of the test specimen at the first scan location.

10. The machine-implemented method of claim 1, wherein the corrected probe assembly orientation includes tilting the probe assembly in at least one axis.

11. The machine-implemented method of claim 1, wherein the acquiring data indicative of the distance comprises performing an acoustic time-of-flight (ToF) determination.

12. The machine-implemented method of claim 1, wherein, when the first transducer arrangement is in the first scan location, the second transducer arrangement at least partially extends beyond a footprint of the test specimen, to allow acquiring data indicative of the distance between the probe assembly and the surface of the test specimen at an edge of the test specimen.

13. A system for performing non-destructive test (NDT) including compensating for surface variation of a test specimen, the system comprising:
a probe assembly;
a manipulator mechanically coupled with the probe assembly and configured to position and orient the probe assembly;
a processor circuit;
a memory circuit comprising instructions that, when executed by the processor circuit, cause the system to:
position the probe assembly relative to a surface of the test specimen, using the manipulator, to perform scanning;
at a first scan location, acquire data indicative of a distance between the probe assembly and the surface of the test specimen;
determine a corrected probe assembly orientation using the data indicative of the distance; and
at the first scan location, perform an inspection acquisition using the corrected probe assembly orientation, the inspection acquisition separate from an acquisition used for a determination of the distance.

14. The system of claim 13, wherein the probe assembly comprises an acoustic probe assembly, the acoustic probe assembly comprising a first region defining a first transducer arrangement used for acquiring data indicative of the distance between the probe assembly and the surface of the test specimen, and a second region defining a second transducer arrangement using for the inspection acquisition.

15. The system of claim 13, wherein the probe assembly comprises a linear array of electroacoustic transducer elements.

16. The system of claim 13, wherein the instructions comprise instructions to acquire further data indicative of the distance between the probe assembly and the surface at a second scan location.

17. The system of claim 13, wherein the data indicative of the distance comprises data indicative of a surface profile of the test specimen.

18. The system of claim 13, wherein the instructions to acquire data indicative of the distance comprises instructions to perform an acoustic time-of-flight (ToF) determination.

19. The system of claim 13, wherein the manipulator is configured to tilt the probe assembly in at least one axis to establish the corrected probe assembly orientation.

20. A system for performing non-destructive test (NDT) including compensating for surface variation of a test specimen, the system comprising:
a means for positioning a probe assembly relative to a surface of the test specimen;
at a first scan location, a means for acquiring data indicative of a distance between the probe assembly and the surface of the test specimen using a first transducer arrangement of the probe assembly;
a means for determining a corrected probe assembly orientation using the data indicative of the distance; and
at the first scan location, a means for performing an inspection acquisition using a second transducer arrangement and using the corrected probe assembly orientation, the inspection acquisition separate from an acquisition used for a determination of the distance using the first transducer arrangement.
